(19) United States
(12) Patent Application Publication
(10) Pub. No.: US 2010/0130436 A1
(43) Pub. Date: May 27, 2010

(54) CO-ADMINISTRATION OF RANOLAZINE AND CARDIAC GLYCOSIDES

(75) Inventors: Luiz Belardinelli, Palo Alto, CA (US); Kirsten Hoyer, San Jose, CA (US); John Shryock, East Palo Alto, CA (US)

Correspondence Address: CV THERAPEUTICS, INC.;Gilead Palo Alto, Inc., 333 Lakeside Drive, Foster City, CA, 94404, US

(73) Assignee: Gilead Palo Alto, Inc., Foster City, CA (US)

(21) Appl. No.: 12/625,403

(22) Filed: Nov. 24, 2009

Related U.S. Application Data
(60) Provisional application No. 61/117,927, filed on Nov. 25, 2008.

(51) Int. Cl. A61K 31/704; A61K 31/70; A61P 9/00

(52) U.S. Cl. 514/26; 514/23

(57) ABSTRACT

The present invention relates to a method for reducing the toxicity of cardiac glycosides comprising the coadministration of a therapeutically effective amount of cardiac glycoside and a therapeutically effective amount ranolazine. This invention also relates to pharmaceutical formulations that are suitable for such combined administration.

### CROSS-REFERENCE TO RELATED APPLICATIONS

This application claims priority to U.S. Provisional Patent Application Ser. No. 61/117,927, filed Nov. 25, 2008, the entire disclosure of which is hereby incorporated by reference.

### FIELD OF THE INVENTION

The present invention relates to method of reducing toxicity of cardiac glycosides by the co-administration of ranolazine. The method finds utility in the treatment of cardiovascular disease, particularly heart failure and atrial fibrillation. This invention also relates to pharmaceutical formulations that are suitable for such combined administration.

### BACKGROUND OF THE INVENTION

Cardiac glycosides such as ouabain and digitalis glycosides have been commonly used in treatment of heart failure, but their therapeutic window is narrow. Ouabain inhibits the sodium-potassium ATPase (sodium pump), leading to an increase in the intracellular sodium concentration ([Na+]i), and, via Na—Ca exchange, the intracellular Ca2+-concentration ([Ca2+]i), thereby enhancing myocardial contractility. Ouabain intoxication is caused, at least in part, by an overload in [Ca2+]i, resulting in diastolic dysfunction and arrhythmias.

Ranolazine (RAN), a novel antianginal drug, has been shown to inhibit the late sodium current (late INa) in the heart without clinically significant changes in heart rate or blood pressure. It has been recently discovered that RAN attenuates the toxic effect of ouabain by reducing Na+ influx and its sequelae of Na/Ca overload and increased energy demand. These results establish that a therapeutically effective dose of RAN reduced the toxicity of cardiac glycosides such as ouabain without reduction of its positive inotropic effect.

### SUMMARY OF THE INVENTION

In one aspect of the invention a method is provided for reducing the toxicity of cardiac glycosides by the co-administration of a therapeutically effective amount of ranolazine. The two agents may be administered separately or together in separate or a combined dosage unit. If administered separately, the ranolazine may be administered before or after administration of the cardiac glycoside but typically the ranolazine will be administered prior to the cardiac glycoside.

In another aspect of the invention a method for reducing the undesirable side effects of cardiac glycosides is presented. The method comprises coadministration of a therapeutically effective dose of cardiac glycoside and a therapeutically effective dose of ranolazine. As before, the two agents may be administered separately or together in separate or a combined dosage unit. If administered separately, the ranolazine may be administered before or after administration of the cardiac glycoside but typically the ranolazine will be administered prior to the cardiac glycoside.

### SUMMARY OF THE FIGURES

FIG. 1 depicts the effect of ouabain on the ventricular pressure in the absence of ranolazine is shown here in this representative pressure tracing of an isolated heart as determined in Example 1. The x-axis is the time scale, the y-axis the Left ventricular pressure in mmHg. The systolic pressure is 101 mmHg during BI conditions and delivery of vehicle. After a short delay upon infusion with ouabain we observed a significant increase in the systolic pressure up to 140 mmHg. This positive inotropic effect was followed by the toxic effect of ouabain seen as an increase in EDP (from 8 to 45 mmHg) and a decrease in SP indicating negative inotropy interrupted by episodes of cardiac stand still. During the 20 min wash out period the systolic function of the heart was reduced compared to baseline conditions, e.g. the systolic pressure at 70 mmHg and the diastolic function still increased—here at 34 mmHg.

FIG. 2 is a representative record of left ventricular contractility of a heart pretreated with 3.3 uM Ran as described in Example 1. During Ranolazine delivery a slight drop of systolic pressure could be observed. However, the maximal inotropic effect of ouabain was the same as in the ouabain alone treated hearts. The toxic effect of ouabain was less pronounced, i.e. there were fewer episodes of cardiac stand still.

FIG. 3 illustrates that hearts w/5 uM Ran show also a transient decrease in SP but the maximal positive inotropic effect of ouabain was not reduced. Furthermore, a decrease is demonstrated in the ouabain-induced negative inotropy and hardly any episodes of cardiac stand still.

FIG. 4 This is a representative record of left ventricular contractility of a heart pretreated with 10 uM Ran which showed stable contractile function over the duration of the experiment.

FIG. 5 Presents the positive inotropic effect of ouabain in the absence and presence of ranolazine, which was not changed but the toxic effect of ouabain was reduced by ranolazine in a dose dependent manner.

FIG. 6 shows that ranolazine has no influence on the maximal positive inotropic effect of ouabain as pointed out in this bar graph for the rate pressure product (RPP). During baseline conditions the RPP is at 26,845±169 mmHg/min, the same as during delivery with vehicle. It significantly increased with ouabain and is markedly decreased during the wash out period compared to baseline. The hearts pretreated with 10 uM ranolazine show a significant decrease of RPP of about 20%. However, upon infusion with ouabain the RPP increase reached the same value as in the absence of ranolazine. Note that during the wash out period RPP was very low after treatment with ouabain alone, and was improved in the presence of ranolazine. Furthermore, Ran did not affect the ability of ouabain to significantly increase the pos dP/dt.

FIG. 7 illustrates the effects of ranolazine to reduce the toxicity of ouabain asdemonstrated in this bar graph depicting the end diastolic pressure (EDP). The EDP is set at 7.3±0.6 mmHg for the baseline conditions with no changes during vehicle delivery but we see a huge increase with ouabain-treatment which does not really recover during the wash out period. However, due to ranolazine the increase in EDP was markedly attenuated at concentrations of 5 and 10 μM and during the wash out period the values were not different from the basal conditions. Similarly, developed pressure was significantly decreased in the ouabain-only treated hearts whereas it was increased when hearts were treated with 10 uM ranolazine.

FIG. 8: Presents the effects of ouabain, ranolazine (Ran), and TTX on intracellular Na⁺-concentration ([Na⁺]ᵢ) measured by ²³Na-NMR spectroscopy of the guinea pig isolated heart. Panel A, shows a typical ²³Na spectrum in which the extracellular Na resonance (Naₑ) was shifted to the left by 1.8 ppm in the presence of the shift reagent Na₅TmDOTP (3.5 mmol/L) compared to the intracellular Na resonance (Naᵢ). Panel B is a stacked plot of Naᵢ resonances obtained every 2 min during control perfusion (10 min) and during perfusion with 10 μmol/L ranolazine (10 Ran, 30 min). Panel C shows the effect of 0.75 μmol/L ouabain (n=3-4) on [Na⁺]ᵢ. Ran (3 and 10 μmol/L, pooled data, n=6) partially reversed the effect of ouabain when administered at 40 min. *P<0.05 vs. ouabain alone at times 66-70 min. Panel D shows the effects of 1.3 μmol/L ouabain on [Na⁺]ᵢ in the absence (□, n=6) and presence of either 10 μmol/L Ran (□, n=8) or 1 μmol/L TTX (□, n=5). Timeline: 1—control, 2—vehicle, Ran or TTX pretreatment, 3—ouabain±drug, 4—washout. *P<0.001 between plateau values of [Na⁺]ᵢ in absence vs. presence of Ran or TTX, †P<0.05, Ran vs. TTX. Panel E presents the values (mean±SEM, n=4-8) of area under the curve (AUC) of [Na⁺]ᵢ during 0-60 min exposures to 1.3 μmol/L ouabain in absence and presence of either Ran (3 and 10 μmol/L) or TTX (0.5 and 1 μmol/L) *P<0.001 vs. 1.3 μmol/L ouabain alone, †P<0.05 vs. 10 Ran.

FIG. 9 demonstrates how ouabain increases late sodium current (late I_{Na}) in guinea pig isolated ventricular myocytes. Panels A and B show the effect of 1 μmol/L ouabain (Ouab) to increase late I_{Na} in a patch-clamped myocyte which is partially reversed by either ranolazine (Ran, 10 μmol/L) or TTX (3 μmol/L). Current traces a-e were successively recorded from a single myocyte. The effect of TTX was reversible upon washout (not shown). Panel C is a summary of effects of Ouab, Ran and TTX on late I_{Na} (n=6-8 myocytes); * and ** P<0.001 vs. control and ouabain alone, respectively.

FIG. 10 depicts how intracellular applications (via the patch pipette) of either KN-93 (10 μmol/L) or EGTA (1 mmol/L), but not KN-92 (10 μmol/L), attenuated the effect of ouabain (1 μmol/L) to increase late I_{Na}. Panel A shows changes of late current amplitude (nC) in each of 4 individual myocytes during a 10-min treatment with ouabain in the absence (control) and presence of KN-92, KN-93, or EGTA. Panel B presents records of late I_{Na} recorded from the 4 cells shown in panel A, at the beginning (0 min) and end (10 min) of an experiment. Dotted line indicates zero current. Calibration bars apply to all records. Panel C is a summary of effect of ouabain (bars represent mean±SEM of data from 6-7 myocytes) on late I_{Na} (pC/pF) recorded at beginning (0 min) and end (10 min) of drug exposures as depicted in panel A. *P<0.01 vs. 0 min. NS, P>0.05 vs. 0 min. Panel D is a comparison of increases of late I_{Na} caused by 1 μmol/L ouabain in the absence (Ctrl) and presence of either KN-92, KN-93, or EGTA, expressed as % of baseline (0 min) current. NS, P>0.05 vs. control; *P<0.01 vs. control and KN-92.

FIG. 11 graphically illustrates the ouabain-induced changes in concentrations of energy-related phosphates measured by ³¹P-NMR spectroscopy of guinea pig isolated hearts. Panel A presents a representative ³¹P-NMR control spectrum. Peak assignments from left to right: phosphomonoesters (PME), extracellular inorganic phosphate (ₑₓPi), intracellular Pi, phosphocreatine (PCr), and □-, □- and □-phosphorus atoms of ATP. Panels B-C, illustrate the changes of ATP, PCr, Pi and intracellular pH (pHᵢ) during exposure to 0.75 μmol/L ouabain (arrow) in the absence or presence of 10 μmol/L ranolazine. Values of ATP and PCr are expressed relative to concentrations measured at time 0 in the presence of vehicle or 10 μmol/L ranolazine. *P<0.05 compared to ouabain alone; ‡P<0.05 for 20-80 min values vs. control (0 time, 100%); ‡P<0.04 vs. control (0 time); #P<0.05 for all ranolazine vs. all ouabain, Wilcoxon's rank sum test. Panel D shows representative stacks of sequential averaged spectra depicting Pi, PCr, and [□-P]-ATP resonances during control (1), ±10 μmol/L ranolazine (2), 0.75 μmol/L ouabain-treatment±10 μmol/L ranolazine (3), and washout periods (4). Panel E is a bar graph showing calculated chemical free energy from ATP hydrolysis (|□G_{˜ATP}|) after 10-min exposures to either no drug (control) or 10 μmol/L ranolazine (Ran alone), and after 60-min exposures to 0.75 μmol/L ouabain in the absence and presence of 10 μmol/L ranolazine. *P<0.05 vs. control, **P<0.001 vs. all groups. Values are means±SEM of data from 5 experiments.

FIG. 12 graphically illustrates the effect of ouabain (0.75 μmol/L) on left ventricular (LV) developed pressure of the guinea pig isolated, electrically-paced heart, in the absence (Panel A) and presence of ranolazine (Ran, 3, 5, and 10 μmol/L; Panels B, C, D, respectively). Records from four representative experiments are shown. Shown to the right of each record are expanded portions of the record at the points indicated by a, b, and c (arrows). The experimental treatment protocol is shown above each record. Ctrl, control (no drug); V, vehicle; Wash, drug washout.

FIG. 13 illustrates the proposed mechanism of the cellular effects of late sodium current (late I_{Na}) and Ca²⁺-calmodulin-dependent protein kinase II (CaMKII) inhibitors (TTX and ranolazine, and KN-93, respectively), and the Ca²⁺-chelator EGTA on ion homeostasis when Na⁺, K⁺-ATPase activity is inhibited by ouabain. [Na⁺]ᵢ and [Ca²⁺]ᵢ, intracellular sodium and calcium concentrations, respectively; NCX, sodium/calcium exchanger.

### DETAILED DESCRIPTION OF THE INVENTION

### Definitions and General Parameters

As used in the present specification, the following words and phrases are generally intended to have the meanings as set forth below, except to the extent that the context in which they are used indicates otherwise.

“Optional” or “optionally” means that the subsequently described event or circumstance may or may not occur, and that the description includes instances where said event or circumstance occurs and instances in which it does not.

“Parenteral administration” is the systemic delivery of the therapeutic agent via injection to the patient.

The term “therapeutically effective amount” refers to that amount of a compound of Formula I that is sufficient to effect treatment, as defined below, when administered to a mammal in need of such treatment. The therapeutically effective amount will vary depending upon the specific activity of the therapeutic agent being used, the severity of the patient's disease state, and the age, physical condition, existence of other disease states, and nutritional status of the patient. Additionally, other medication the patient may be receiving will effect the determination of the therapeutically effective amount of the therapeutic agent to administer.

The term “treatment” or “treating” means any treatment of a disease in a mammal, including:
(i) preventing the disease, that is, causing the clinical symptoms of the disease not to develop;
(ii) inhibiting the disease, that is, arresting the development of clinical symptoms; and/or
(iii) relieving the disease, that is, causing the regression of clinical symptoms.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

### Non-standard Abbreviations and Acronyms

ATX-II sea anemone toxin-II
AUC area under the curve
[ ]ᵢ intracellular concentration of a metabolite or ion
CaMKII Ca²⁺-calmodulin-dependent kinase II
late I_{Na} late sodium current (=persistent sodium current)
LVEDP LV end diastolic pressure
LVSP LV systolic pressure
[Na⁺]ᵢ intracellular sodium concentration
NCX sodium/calcium exchanger
NMR nuclear magnetic resonance
PCr phosphocreatine
pHᵢ intracellular pH
Pi intracellular inorganic phosphate
sodium pump Na⁺, K⁺-ATPase
SR sarcoplasmatic reticulum
TTX tetrodotoxin
   |ΔG_{˜ATP}| absolute value of free energy of ATP hydrolysis

### The Method of the Invention

The present invention relates to methods of reducing toxicity of cardiac glycosides. The method comprises coadministration of a synergistic therapeutically effective amount of the glycoside and therapeutically effective amount ranolazine. The two agents may be administered separately or together in separate or a combined dosage unit. If administered separately, the ranolazine may be administered before or after administration of the glycoside but typically the ranolazine will be administered prior to the glycoside

Cardiac glycosides have been used for centuries in treatment of heart diseases. Their beneficial effects are the increase of myocardial contractility in patients with heart failure, and their ability to reduce the atrioventricular node conduction, hence decreasing the ventricular rate as a treatment for atrial arrhythmia. However, the margin between the therapeutic and toxic dose is small. An overdose may result in mechanical dysfunction such as negative inotropy and increased diastolic tension as well as in electrical dysfunctions such as arrhythmia. Ouabain is the cardiac glycoside we used in this study (Strophanthidin, Strodival-D).

Ranolazine, a new anti-anginal/anti-ischemic drug, was approved for treatment of chronic (stable) angina in the United States in January 2006. Ranolazine inhibits the late portion of the sodium current. The sodium current can be simplistically divided into two components, the peak and the late sodium current. Ranolazine does not inhibit the peak sodium current which is responsible for the upstroke of an action potential (AP) but reduces the late sodium current that occurs during the plateau phase of the AP. The late sodium current is normally small but because it flows throughout the entire AP plateau, its contribution to Na+-influx is equivalent to that of peak Ina. Late I_{Na} is increased by congenital gain-of-function mutations in the sodium channel gene SCN5A, by ischemia, heart failure, and by other acquired channelopathies. Much evidence indicates that reduction of the late sodium current by ranolazine reduces sodium entry and sodium-induced Ca-overload in myocytes. Ranolazine has no or little direct effect on Na, K-ATPase, NCX (inward sodium calcium exchanger current) or calcium channels in the therapeutic range.

While not wishing toe be bound by theory, the method of the invention is based on the premise that ranolazine attenuates the sodium-calcium-overload caused by ouabain. A model of sodium-calcium homeostasis is presented here: intracellular sodium homeostasis is determined by the balance between sodium influx during the peak and late phases and sodium efflux by the sodium-potassium ATPase activity. Sodium can also be exchanged with calcium through the sodium calcium exchanger increasing the intracellular calcium concentration, and resulting in an increase in contractility.

Inhibition of the Na,K-ATPase by ouabain causes the intracellular sodium concentration to rise, which induces an increase of NCX activity in the reverse mode, resulting in an increase of intracellular Ca2+ concentration. And this leads to the positive inotropic effect of ouabain, the enhanced contractility. Excessive inhibition of Na, K-ATPase causes a further increase of intracellular Na and Ca, reaching the limits to the ability of cardiac cells to handle the overload and therefore, inducing the toxic effects such as impaired contractility and abnormal electrical activity.

Ranolazine reduces ouabain toxicity by inhibiting the late sodium current and sodium influx and therefore, restores sodium and calcium homeostasis in the presence of ouabain, while maintaining the desired beneficial effect of ouabain, (the positive inotropy).

Ranolazine and the cardiac glycoside may be given to the patient in either single or multiple doses by any of the accepted modes of administration of agents having similar utilities, for example as described in those patents and patent applications incorporated by reference, including buccal, intra-arterial injection, intravenously, intraperitoneally, parenterally, intramuscularly, subcutaneously, orally, or via an impregnated or coated device such as a stent, for example, or an artery-inserted cylindrical polymer.

One mode for administration is parental, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the component in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of the components. Administration may be via capsule or enteric coated tablets, or the like. In making the pharmaceutical compositions that include ranolazine and at least one co-administered agent, the active ingredients are usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compounds, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained or delayed release of the active ingredient after administration to the patient by employing procedures known in the art. As discussed above, given the reduced bioavailabity of ranolazine, sustained release formulations are generally preferred. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345.

The compositions are preferably formulated in a unit dosage form. The term “unit dosage forms” refers to physically discrete units suitable as unitary dosages for human subjects and other mammals, each unit containing a predetermined quantity of the active materials calculated to produce the desired therapeutic effect, in association with a suitable pharmaceutical excipient (e.g., a tablet, capsule, ampoule). The active agents of the invention are effective over a wide dosage range and are generally administered in a pharmaceutically effective amount. It will be understood, however, that the amount of each active agent actually administered will be determined by a physician, in the light of the relevant circumstances, including the condition to be treated, the chosen route of administration, the actual compounds administered and their relative activity, the age, weight, and response of the individual patient, the severity of the patient's symptoms, and the like.

For preparing solid compositions such as tablets, the principal active ingredients are mixed with a pharmaceutical excipient to form a solid preformulation composition containing a homogeneous mixture of a compound of the present invention. When referring to these preformulation compositions as homogeneous, it is meant that the active ingredients are dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. Ranolazine and the co-administered agent(s) can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I

### Background

The follow experiment was conducted to determine if ranolazine as a late sodium current inhibitor attenuates the sodium-calcium-overload caused by ouabain: intracellular sodium homeostasis is determined by the balance between sodium influx during the peak and late phases and sodium efflux by the sodium-potassium ATPase (Na,K-ATPase) activity. Sodium can also be exchanged with calcium through the sodium calcium exchanger increasing the intracellular calcium concentration, and resulting in an increase in contractility. Inhibition of the Na,K-ATPase by ouabain causes the intracellular sodium concentration to rise, which induces an increase of NCX activity in the reverse mode, resulting in an increase of intracellular Ca2+ concentration. This leads to the positive inotropic effect of ouabain (enhanced contractility). Excessive inhibition of Na, K-ATPase causes a further increase of Na and Ca, reaching the limits to the ability of cardiac cells to handle the overload and therefore, inducing the toxic effects such as impaired contractility and abnormal electrical activity.

### Methods:

### Isolated Perfused Heart Preparation

Duncan Hartley guinea pigs (250-350 g) were anesthetized, hearts isolated, and perfused in the isovolumic Langendorff mode (constant pressure at 60 mmHg). Systolic, diastolic function and contractility were recorded via a fluid filled balloon inserted into the left ventricular and connected to a pressure transducer. A modified Krebs-Henseleit (KH) buffer (37° C., pH 7.4) contained (in mmol/L) 118 NaCl, 4.8 KCl, 1.75 CaCl₂, 1.2 MgSO₄, 0.5 EDTA, 25 NaHCO₃, 1.2 KH₂PO₃, 5.5 glucose, 2 pyruvate, and was oxygenated with 95% O₂/5% CO₂. Hearts were paced at 5 Hz over the whole time of the experiments.

### ²³Na- and ³¹P-NMR Measuring [Na⁺]ᵢ and High-Energy Metabolites

²³Na Nuclear Magnetic Resonance Free Induction Decay (NMR FIDs) signals were acquired at 105.5 MHz using a 5-kHz spectral width and 1600 data points. For each heart the 90° pulse width was determined (28-32 μs) after which 590 FIDs were accumulated with a repetition time of 0.2 s for 2 min. FIDs were Fourier transformed after Gaussian multiplication. For distinguishing extracellular and intracellular sodium, 3.5 mmol/L of the shift reagent TinDOTP⁵⁻ (thulium[III] 1,4,7,10-tetraazacyclododecane-N,N′N″N″-tetra[methylene-phosphonate], sodium salt) and because of its calcium chelator characteristics additional CaCl₂ had been added to the KH buffer (SRX buffer).

Quantification was performed relative to the peak area of the reference resonance. The reference capillary contained a known amount of 500 mM Na⁺ and 10 mM TmDOTP⁵⁻.

³¹P-NMR FIDs were acquired at 161.4 MHz, averaging 125 FIDs over 5 min (60° pulse, 2.4-s recycle time). ³¹P-resonance areas were quantified using the Bayesian analysis software (Washington University, St. Louis, Mo.). The respective cytosolic concentrations were determined and the pHi was calculated from the shift between the phosphocreatine and inorganic phosphate peak.

### Experimental Protocols

The protocols were designed to study the effect of late sodium current inhibitors on the isolated heart in conditions in which the sodium pump is inhibited by the cardiac glycoside ouabain:

1. Protocol used for assessment of isovolumic contractile performance and high energy metabolites (P-1):Hearts were perfused with KH buffer until obtaining a stable baseline (equilibration period of 20-30 min), which was recorded for 10 min, followed by 10 min pretreatment of I_{NaL} inhibitors in different concentrations (e.g. 3.3, 5, 10 μmol/L ranolazine, and 0.5, 1 μmol/L tetrodotoxin [TTX]) or vehicle after which an addition of 0.75 μmol/L ouabain was delivered for 60 min, followed by a washout period of both drugs for 20 min,

2. Protocol used for measuring [Na⁺]ᵢ was modified as followed: After obtaining a stable baseline with KH buffer the buffer was switched to the SRX buffer containing the shift reagent. Within 20 min the peak separation of extra- and intracellular sodium was satisfactory and a new baseline was achieved and recorded for 10 min. From this point on the first protocol was followed using an ouabain concentration of 1.3 μmol/L.

### Results:

[Na⁺]ᵢ accumulation and high-energy phosphate content were studied with ²³Na- and ³¹P-NMR spectroscopy, respectively, as well as contractile function, in male guinea-pig hearts paced at 5 Hz during isovolumic Langendorff perfusion. Hearts were pretreated with vehicle or RAN (10 μM) for 10 min, then exposed to ouabain (0.75-1.3 μM) for 60 min in the continued presence of vehicle or RAN, followed by drug washout. Ouabain induced a transient increase in contractile function, which then declined due to its toxic effect. RAN did not reduce the positive inotropic response to ouabain.

Furthermore, RAN did not change cardiac [Na⁺]ᵢ during normal perfusion, but reduced the [Na⁺]ᵢ accumulation during ouabain treatment (figure, mean±SEM, P<0.01). In the ouabain-alone treated hearts, ATP and phosphocreatine (PCr) contents were reduced by 64 and 59%, respectively (figure, mean±SEM, P<0.001), the intracellular content of inorganic phosphate (Pi) was increased 4.8 fold, and pH declined from 7.14 (baseline) to 7.07. In contrast, ATP and PCr were preserved during pretreatment with RAN and decreased by only 20% (P<0.001) after 60 min of ouabain treatment; Pi increased only slightly (14%, P<0.05) and pH remained constant.

Furthermore, in the hearts treated with ouabain alone, an increase in end diastolic pressure and several episodes of cardiac standstill were observed; mechanical dysfunction was not observed in hearts treated with ouabain+RAN. Thus, RAN not only attenuated the toxic effects of ouabain on [Na⁺]ᵢ accumulation and contraction, it also preserved the high-energy phosphate content of the heart.

### Example 2

### Background

The present example illustrates how ouabain increased late I_{Na} in guinea pig myocytes, and inhibition of late I_{Na} attenuated the ouabain-induced Na⁺ overload and metabolic, electrical, and mechanical dysfunction in the guinea pig isolated heart and papillary muscle. The number of conditions now known to be associated with an enhancement of late I_{Na} includes inherited channelopathies (e.g., mutations in SCN5A), heart failure, ischemia/reperfusion, hypoxia, myocardial remodeling, activation of CaMKII, oxidizing agents (e.g., H₂O₂), toxins (e.g., ATX-II), and the cardiac glycoside ouabain (this study).

The last 10 to 15 years has thus been witness to remarkable growth in understanding of the number of conditions that enhance late I_{Na} indicating its key position in pathologies of cellular function. Calcium overload is common to many conditions in which late I_{Na} is increased, and the CaMKII inhibitor KN-93 attenuated the effect of ouabain to increase late I_{Na} in myocytes in this study. This suggests that the cardioprotective actions of ranolazine and TTX depend ultimately on their ability to contribute to Ca²⁺ homeostasis. Because late I_{Na} itself is a cause of calcium overload, there appears to be a positive feedback loop between increases of late I_{Na} and increases of intracellular calcium. Inhibitors of late I_{Na} as well as reduction of Ca²⁺ overload and inhibition of pathological calcium-dependent pathways may interrupt this feedback and protect the heart from Na⁺/Ca²⁺ overload caused by a cardiac glycoside.

### Methods

### Guinea Pig Isolated Perfused Heart Preparation

Guinea pigs were anesthetized (180 mg/kg sodium pentobarbital, i.p.) and hearts were isolated and perfused in the isovolumic Langendorff mode at a constant pressure of 60 mmHg with a modified Krebs-Henseleit (KH) buffer (37° C., pH 7.4) containing (in mmol/L) 118 NaCl, 4.8 KCl, 1.75 CaCl₂, 1.2 MgSO₄, 0.5 EDTA, 25 NaHCO₃, 1.2 KH₂PO₄, 5.5 glucose, 2 pyruvate, oxygenated with 95% O₂/5% CO₂. Contractile function of paced hearts (5 Hz) was measured as previously described.²⁸

### ²³Na and ³¹P Nuclear Magnetic Resonance (NMR) Spectroscopy for Measuring [Na⁺]ᵢ and High-Energy Metabolites in Guinea Pig Isolated Hearts

NMR measurements were performed in a Varian Inova spectrometer (Varian, Palo Alto, Calif.). For ²³Na NMR, 590 free induction decay (FIDs) signals were acquired at 105.5 MHz and averaged over 2 min (90° pulse, 0.2 s recycle time). To distinguish intracellular from extracellular sodium, 3.5 mmol/L of the shift reagent Na₅TmDOTP was added to the KH buffer. To determine _{[Na}⁺], the peak areas of ²³Na signals were compared to the peak area of Na⁺ internal reference standard. ²⁹

For ³¹P-NMR, 125 FIDs were acquired at 161.4 MHz and averaged over 5 min (60° pulse, 2.4 s recycle time). Cytosolic concentrations of ATP, phosphocreatine (PCr), and inorganic phosphate (Pi) were determined according to Shen et al. (2001).²⁸ ΔG_{˜ATP}, the energy released from ATP hydrolysis, was calculated as previously described, assuming a total creatine concentration of 22.0 mmol/L of the perfused guinea pig heart.³⁰

### Experimental Protocols

### Contractile and High-Energy Phosphate Measurements

Hearts were perfused with KH buffer until stable (LVDevP or ³¹P NMR signal), then pretreated for 10 min with either ranolazine (3, 5, 10 μmol/L), tetrodotoxin (0.5, 1 μmol/L, TTX) or vehicle (0.02% DMSO in KH buffer), then exposed to 0.75 μmol/L ouabain in the continued presence of ranolazine, TTX or vehicle for 60 min.

### Measurement of [Na⁺]ᵢ

Hearts were stabilized in KH buffer then perfused with a modified KH buffer containing the shift reagent Na₅Tm[DOTP]. Each heart was exposed to either: (1) 10 μmol/L ranolazine for 30 min; (2) 0.75 μmol/L ouabain for 40 min, and then to ouabain in the absence or presence of ranolazine (3, 10 μmol/L) for an addition at 30 min; (3) ranolazine (3, 10 μmol/L), TTX (0.5, 1 μmol/L) or vehicle (0.02% DMSO) for 10 min followed by exposure to 1.3 μmol/L ouabain for 60 min in the continued presence of ranolazine, TTX or vehicle. The higher concentration of ouabain (1.3 mol/L) used to cause greater and faster cellular Na⁺ loading than achieved with 0.75 μmol/L ouabain. After treatment all drugs were washed out for 20 min.

### Isolation of Ventricular Myocytes and Electrophysiological Recordings

Single guinea pig ventricular myocytes were isolated using standard enzymatic procedures as described previously. ²⁵ Transmembrane Na⁺ currents were measured, using the whole-cell patch-clamp technique. The recording pipettes had a resistance of 2-3 M□ when filled with a solution containing (in mmol/L) 120 Cs-aspartate, 20 CsCl, 1 MgSO₄, 4 Na₂ATP, 0.1 Na₃GTP, and 10 HEPES, pH 7.2. Late I_{Na} was activated using 300-ms voltage-clamp pulses from −90 to −50 mV at a frequency of 0.16 Hz. Transmembrane current during the last 100 ms of depolarizing pulse was integrated and expressed as nano- or picocoulombs (nC or pC). Cell membrane capacitance was minimized using the amplifier, and values of capacitance compensation in picofarads (pF) were used to normalize the integrated current to the magnitude of the membrane capacitative current (pC/pF). During experiments, myocytes were superfused with a bath solution (36° C.) containing (in mmol/L) 135 NaCl, 4.6 CsCl, 1.8 CaCl₂, 1.1 MgSO₄, 0.01 nitrendipine, 0.1 BaCl, 10 glucose and 10 HEPES, pH 7.4. KN-93, KN-92 and EGTA included in the recording pipette solution to achieve intracellular application, whereas ouabain, TTX, and ranolazine were applied extracellularly via the bath solution.

### Statistics

Results are expressed as mean±SEM. Data were analyzed by one-way analysis of variance (ANOVA) or ANOVA with repeated measures (Statistica 8.0, Stat Soft, Inc., Tulsa, Okla., USA), followed by a post hoc test (e.g., Tukey's test) when significant differences were observed. Calculation of the area under the curve was performed with GraphPad Prism 5.01 (GraphPad Software, San Diego, Calif., USA). A p value<0.05 was considered to indicate a significant difference.

### Results

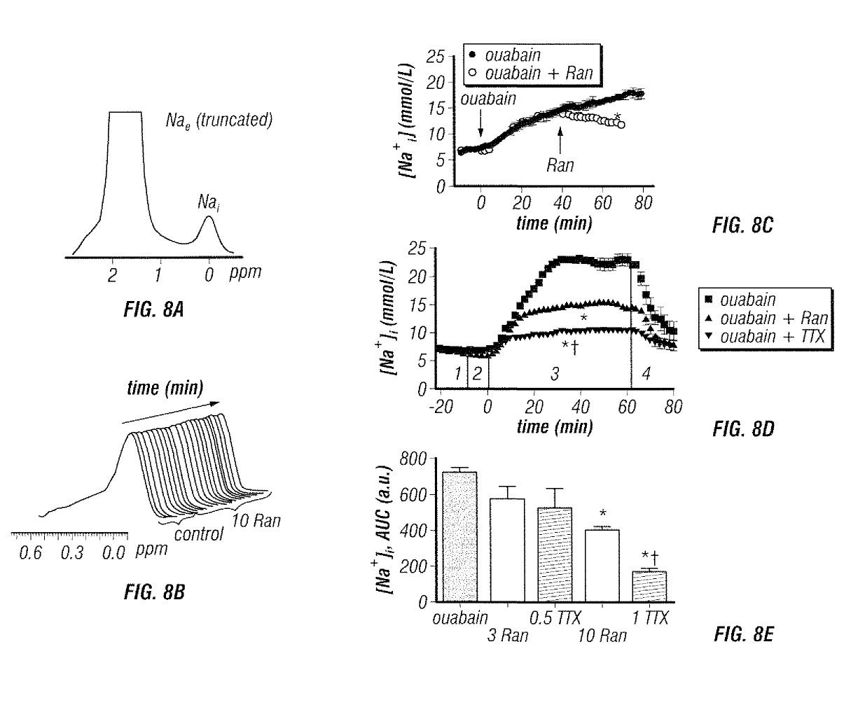

### Changes in [Na⁺]ᵢ During Sodium Pump Inhibition in the Absence and Presence of Ranolazine and Tetrodotoxin (TTX)

[Na⁺]ᵢ of the guinea pig isolated, perfused heart in the absence of drug was 6.9±0.6 mmol/L (n=9; FIG. 8A), as determined by ²³Na NMR spectroscopy. After perfusion of the heart with ranolazine (10 μmol/L) for 30 min, [Na⁺]ᵢ was 6.5±0.4 mmol/L (n=5, P>0.1 vs. control; FIG. 8B). During exposure of hearts to 0.75 μmol/L ouabain for 40 and 76 min in the absence of ranolazine, [Na⁺]ᵢ increased to 14.9±0.9 and 18.1±0.9 mmol/L, respectively (n=4, P<0.001 vs. control, P<0.05 40 vs. 76 min; FIG. 8C). Exposure of hearts to either 3 or 10 μmol/L ranolazine after 40 min of treatment with ouabain alone partially reversed the ouabain-induced increase in [Na⁺]ᵢ (n=6; FIG. 8C). Upon exposure of the heart to a higher concentration of ouabain (1.3 rather than 0.75 μmol/L), [Na⁺]ᵢ increased rapidly by 3.3-fold at 60 min to reach a plateau level of 22.9±0.7 mmol/L (n=6, P<0.001 vs. control; FIG. 8D).

After washout of ouabain for 20 min, [Na⁺]ᵢ was 10.3±1.8 mmol/L (n=5, P<0.001 vs. plateau level, P<0.05 vs. control), indicating that the ouabain effect was at least partially reversible. The 1.3 μmol/L ouabain-induced increase of [Na⁺]ᵢ could be attenuated by treatment of hearts with either ranolazine (10 μmol/L) or TTX (1 μmol/L) for 10 min prior to and during the exposure to ouabain (FIG. 8D,E). During treatment of hearts with 1.3 μmol/L ouabain in the presence of either 10 μmol/L ranolazine (n=8) or 1 μmol/L TTX (n=5), values of [Na⁺]ᵢ reached plateau concentrations of 15.4±0.45 or 10.5±0.3 mmol/L, respectively (both P<0.001 vs. ouabain alone and P<0.05 vs. control). The decrease in the ouabain-induced rise of [Na⁺]ᵢ by ranolazine and TTX was concentration-dependent (FIG. 8E).

To exclude the possibility that ranolazine had a direct effect on the sodium pump, three different ranolazine concentrations (3, 10, 30 μmol/L) were tested in a Na⁺, K⁺-ATPase activity assay³¹ by measuring the ⁸⁶Rb⁺ uptake of A7r5 cells in the presence of ouabain with or without ranolazine. The activity of Na⁺, K⁺-ATPase was inhibited 77% by 1 mmol/L ouabain in the absence (control) of ranolazine. Values of ⁸⁶Rb⁺ uptake were 91±11, 98±2.5, and 93±8.3% of control (activity in presence of ouabain) in the presence of 3, 10, and 30 μmol/L ranolazine, respectively. The results suggest that ranolazine has no measurable effect on sodium pump activity in this assay.

### Ouabain-Induced Late I_{Na}

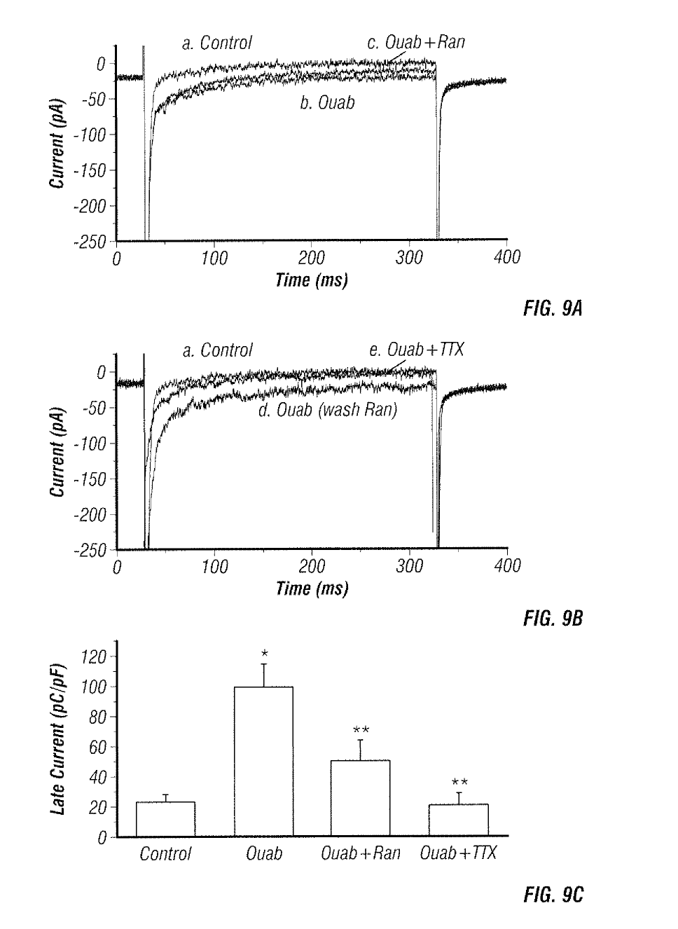

The amplitude of late I_{Na} in guinea pig isolated ventricular myocytes was increased by exposure of cells to ouabain (1 μmol/L). After a 3 to 5-min exposure of myocytes to ouabain, the integrated late I_{Na} (see supplemental material for details) was increased from 23.5±4.9 to 99.6±15.2 pC/pF (n=8, P<0.001; FIG. 9A-C). Ranolazine (10 μmol/L) applied to cells in the continuous presence of ouabain reduced late I_{Na} by 69±9%, from 99.6±15.2 to 50.6±13.6 pC/pF (n=8, P<0.001; FIG. 9A, 9C). In some experiments, after washout of ranolazine, cells were exposed to TTX (3 μmol/L, n=6, FIG. 9B). Ouabain-induced late current was completely inhibited by TTX, to 21.2±7.9 pC/pF (P<0.001), indicating that the late current induced by ouabain was a TTX-sensitive sodium current (e.g., Naᵥ1.5).

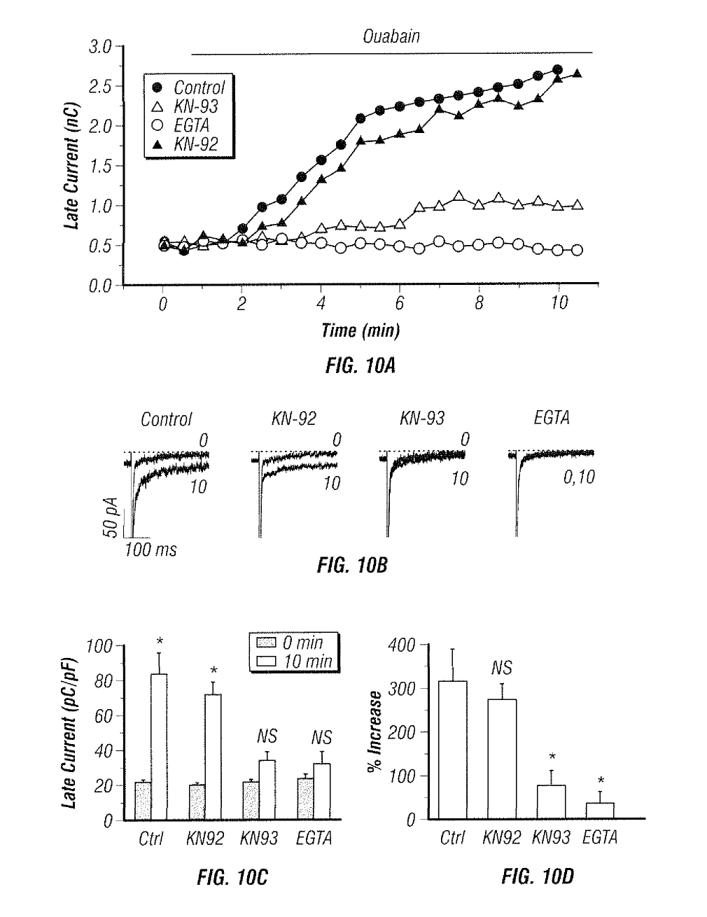

To examine the hypothesis that a Ca²⁺-dependent, CaMKII-mediated mechanism may underlie the effect of ouabain to increase late I_{Na}, cells were incubated with ouabain when either the CaMKII inhibitor KN-93 (10 μmol) or the Ca²⁺ chelator EGTA (1 mmol/L) was dialyzed into them by inclusion in the patch pipette solution. KN-92 (10 μmol/L), an inactive analog of KN-93, was used as a control. Ouabain alone (1 μmol/L, n=6) caused a time-dependent increase of late I_{Na} by 318±74% from 21±2 to 84±12 pC/pF (P=0.003) in 5-10 min (FIG. 10A-10C). In comparison, at the end of a 10-min exposure to ouabain in the presence of intracellular KN-93, late I_{Na} was increased by only 76±35% (from 21±2 to 33±6 pC/pF; n=7, P=0.003 vs. ouabain alone), and at the end of a 10-min exposure to ouabain in the presence of EGTA, late I_{Na} was increased by only 33±28% (from 23±3 to 31±8 pC/pF; n=6, P<0.001 vs. ouabain alone) (FIG. 10D). In contrast, in the presence of KN-92, the increase of late I_{Na} at the end of a 10-min exposure to ouabain was 273±39% (from 20±1 to 72±7 pC/pF; n=6, P>0.05 vs. ouabain alone, and P<0.01 vs. KN-93 or EGTA).

### Changes in Energy-Related Phosphates During Sodium Pump Inhibition in the Absence and Presence of Ranolazine

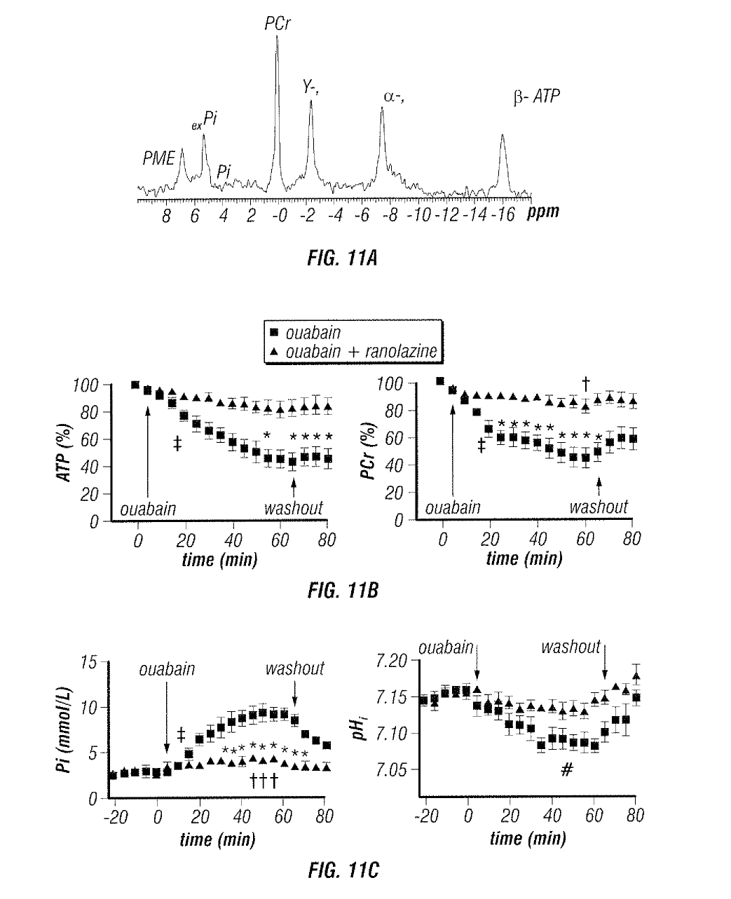

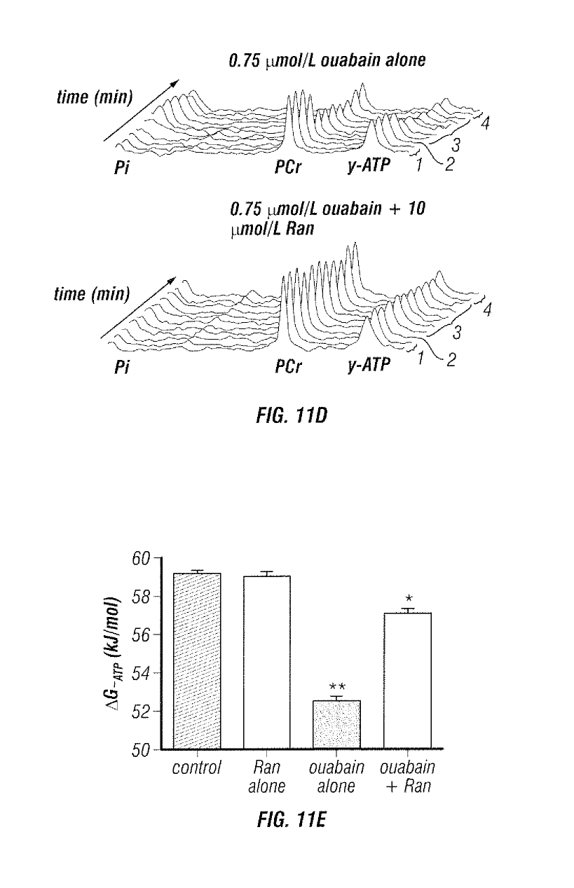

One of the consequences of Na⁺ and Ca²⁺ overload is a mismatch of energy supply and demand. Therefore, we measured changes of energy-related phosphates with ³¹P NMR spectroscopy in ouabain-treated guinea pig isolated, perfused hearts in the absence and presence of ranolazine. Under control conditions [ATP], [PCr], and [Pi] were 9.9±0.3, 13.9±0.5, and 2.9±0.2 mmol/L, respectively (n=10 each; FIG. 11A), and intracellular pH (pHᵢ) was 7.15±0.01 (n=10). Exposure of hearts to ranolazine alone for 10 min did not alter either concentrations of phosphates or pHᵢ. After exposure to 0.75 μmol/L ouabain for 60 min, [ATP] and [PCr] declined by 55±6 and 56±7%, respectively, [Pi] increased by 3.7±0.9-fold (all n=5; FIG. 11B-11D), and pHᵢ declined to 7.07±0.01 (FIG. 11C). Values of pHᵢ and Pi recovered during a 20-min washout period; pHᵢ returned to 7.15 (control) and [Pi] decreased from 9.1±0.5 to 5.8±0.4 mmol/L (P<0.001; FIG. 11C). In hearts treated with 0.75 μmol/L ouabain in the presence of 10 μmol/L ranolazine, [ATP] did not change significantly and [PCr] decreased by only 19±5% from baseline (P<0.05 compared to ouabain alone) after 60 min ouabain treatment (FIG. 11B, 11D). The value of [Pi] increased only slightly (1.3±0.1 times, P<0.04 vs. control), and the fall in pHᵢ was not significant (FIG. 11C), in the presence of ouabain and ranolazine.

Values of [ATP], [PCr] and [Pi] were used to calculate |□G_{˜ATP}|, the energy released from ATP hydrolysis that is available for the ATPase reactions in the cell. The value of |□G_{˜ATP}| was 59.2 kJ/mol in control hearts, and it decreased by 7.2±1.2 kJ/mol (P<0.001) in hearts treated for 60 min with 0.75 μmol/L ouabain (FIG. 10E). In contrast, |□G_{˜ATP}| decreased by only 1.9±0.6 kJ/mol in hearts treated with ouabain in the presence of 10 μmol/L ranolazine (P<0.05 vs. control, P<0.001 vs. ouabain; FIG. 11E).

### Changes in Contractile Function of the Isolated Heart During Ouahain-Induced Sodium Pump Inhibition in the Absence and Presence of Ranolazine or TTX

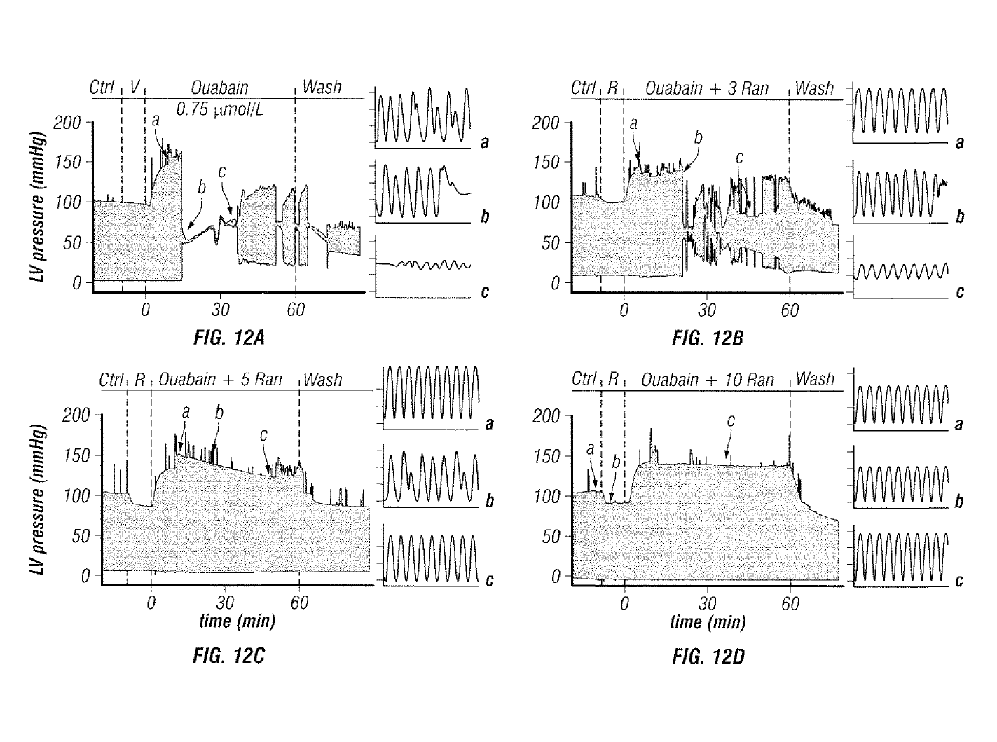

Control (absence of drug) values of left ventricular systolic pressure (LVSP), rate pressure product (RPP: HR×LV developed pressure [LVDevP]), and LV end diastolic pressure (LVEDP) in 5 Hz-paced guinea pig isolated, perfused hearts (n=46) were 96±3 mmHg, 26,845±169 mmHg/min, and 7.3±10.6 mmHg, respectively. Treatment of hearts with 0.75 μmol/L ouabain (n=13) led to an increase of LVSP by 55±5% from 96±2 to 148±6 mmHg (P<0.001) and an increase of RPP by 59±6% from 26,497±664 to 40,676±1159 mmHg/min (P<0.001), followed by episodes of cardiac standstill (with an elevated LVEDP) alternating with periods of rhythmic contraction (FIG. 12A).

LVSP decreased by 8, 16.7 and 15.8% (n=6-8 each) during treatment with ranolazine alone (3, 5, 10 μmol/L, respectively; P<0.05) and by 9 and 18.2% (n=5-6) during treatment with TTX (0.5 and 1 μmol/L, respectively; P<0.04). Ranolazine and TTX (not shown) attenuated the effect of ouabain to cause contractile dysfunction, but without preventing the positive inotropic response to the glycoside (FIG. 12).

Thus, the maximum values of RPP rose to 40,033±1,477; 38,874±2,904; and 41,079±2,097 mmHg/min in the hearts treated with 0.75 union ouabain+3 (n=8), 5 (n=5), or 10 (n=8) μmol/L ranolazine, respectively, and to 35,569±1,850 and 39,182±2,216 mmHg/min in hearts treated with ouabain+0.5 (n=5) or 1 (n=6) μmol/L TTX, respectively. Indications for the toxic effect of ouabain include cardiac standstill and elevated LVEDP. Episodes of cardiac standstill (i.e., absence of a contractile response during continuous electrical pacing at 5 Hz) occurred in 11 out of 13 hearts treated with 0.75 μmol/L ouabain for 60 min, concurrent with a marked elevation of LVEDP (FIG. 12A). Ranolazine and TTX reduced the occurrence of episodes of cardiac standstill caused by ouabain. Of eight hearts treated with 0.75 union ouabain+3 μmol/L ranolazine, four hearts showed episodes of cardiac standstill including elevated LVEDP (FIG. 12B) whereas the remaining four hearts maintained enhanced but irregular contractility.

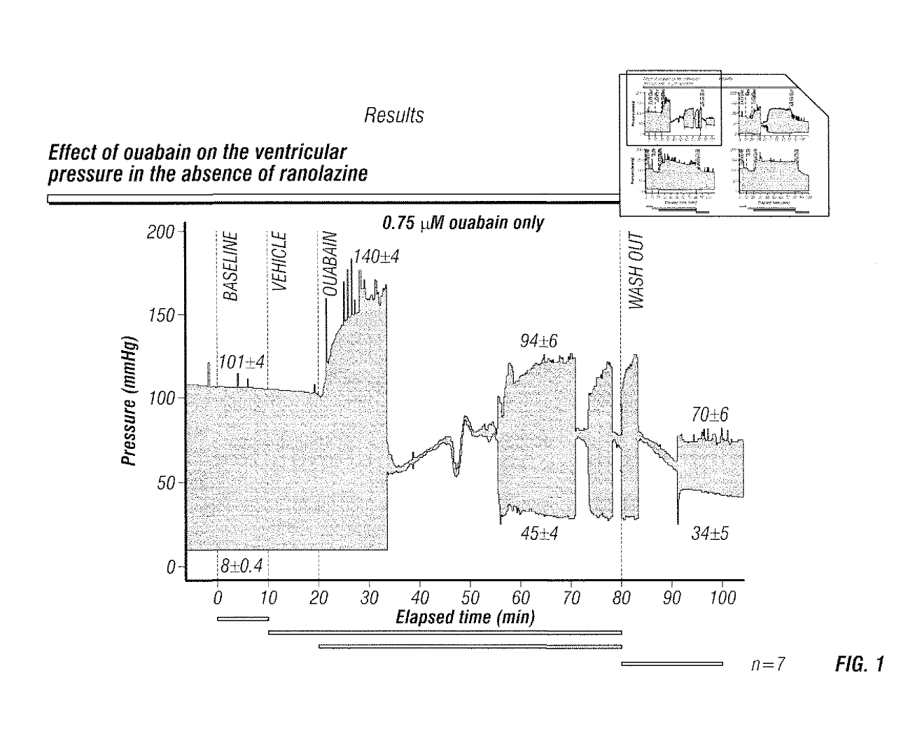

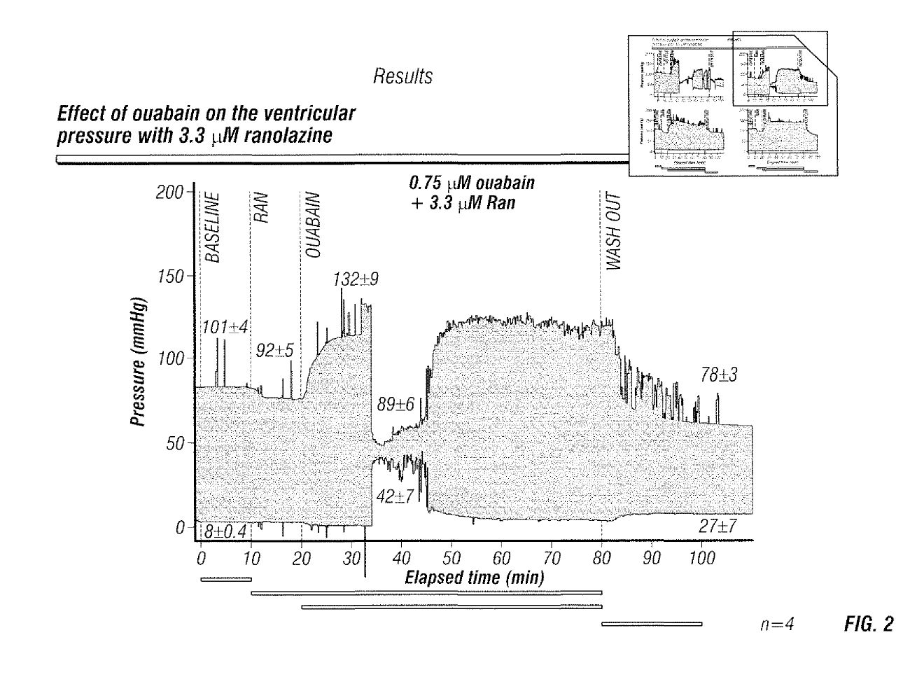

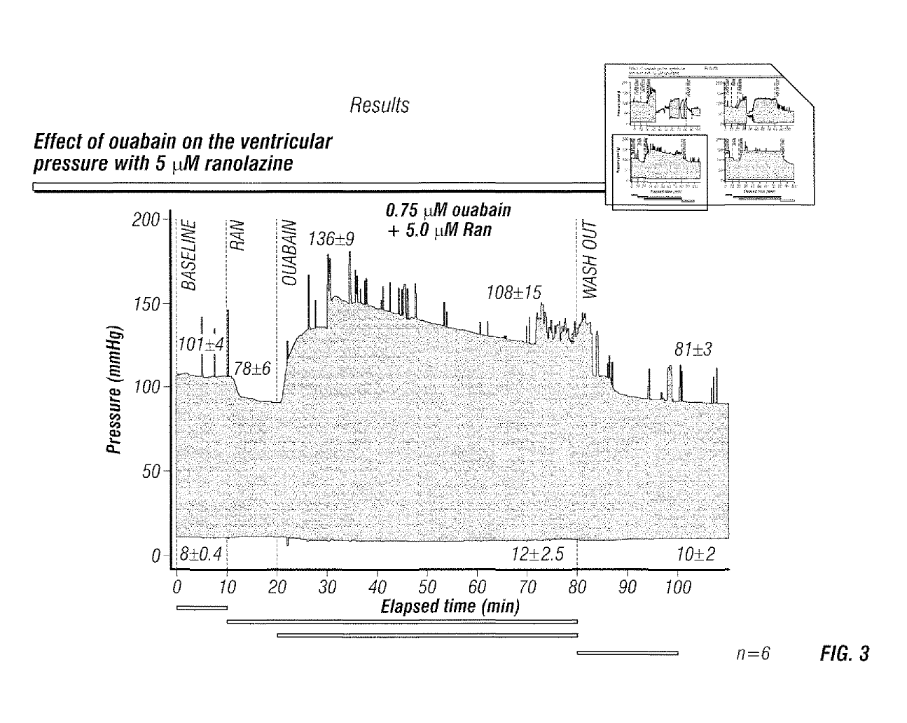

The responses of hearts that were exposed to ouabain in the presence of 0.5 μmol/L TTX were comparable to those exposed to ouabain in the presence of 3 union ranolazine. When the concentration of ranolazine was increased to 5 μmol/L, hearts treated with 0.75 μmol/L ouabain (n=6, FIG. 12C) did not have episodes of cardiac standstill. However, as in hearts treated with 0.75 μmol/L ouabain alone, the positive inotropic response to ouabain was not sustained at a maximal level during the 60-min ouabain exposure in the presence of 5 μmol/L ranolazine, and irregular rhythmic episodes were sometimes observed, although LVEDP was not significantly changed compared to control. In contrast, the positive inotropic effect of ouabain was sustained in hearts treated with either 10 μmol/L ranolazine (n=8, FIG. 12D) or 1 μmol/L TTX (n=6, not shown) throughout the 60-min duration of ouabain exposure, and neither episodes of cardiac standstill nor changes in LVEDP were observed.

Hearts exposed to ouabain in the presence of 5 or 10 μmol/L ranolazine or 1 μmol/L TTX showed better recovery of contractile function after drug washout than hearts treated with ouabain alone. LVDevP at the end of washout was significantly reduced in hearts treated with ouabain alone, ouabain+3 μmol/L ranolazine or +0.5 μmol/L TTX (36.4±5.9, 59.2±5.5, and 44.6±9.5 mmHg, respectively; P<0.05) compared to control (89.1±1.0 mmHg, n=46), whereas in hearts treated with ouabain+5 or 10 μmol/L ranolazine or 1 μmol/L TTX LVDevP was not significantly different from control.

Changes in contractile function measured in papillary muscle preparations during ouabain-induced sodium pump inhibition in the absence and presence of ranolazine confirmed these observations in the intact beating heart.

### Discussion

The results presented here suggest that a reduction of late I_{Na} attenuates sodium accumulation and metabolic, contractile, and electrical dysfunction induced by a cardiac glycoside in the guinea pig isolated, perfused heart and papillary muscles. The cardiac glycoside ouabain markedly increased [Na]ᵢ and [H⁺]ᵢ and decreased [ATP] and [PCr] in the heart. Ranolazine (10 μmol/L) and TTX (1 μmol/L) at concentrations reported to inhibit late I_{Na} (Song et al. Am J Physiol Heart Circ Physiol. 2008; 294:H2031-2039) significantly reduced the rise in [Na]ᵢ and attenuated the losses of [ATP] and [PCr] and the decrease of pHᵢ that were observed in the presence of ouabain alone. Both 5 and 10 μmol/L ranolazine and 1 μmol/L TTX prevented the rise of LVEDP and reduced occurrences of cardiac standstill caused by ouabain in the isolated perfused heart, and attenuated the increase of diastolic tension of isolated guinea pig papillary muscles during ouabain treatment. Thus, a reduction of late I_{Na} is cardioprotective when [Na]ᵢ is elevated as a result of glycoside-induced inhibition of the Na⁺, K⁺-ATPase.

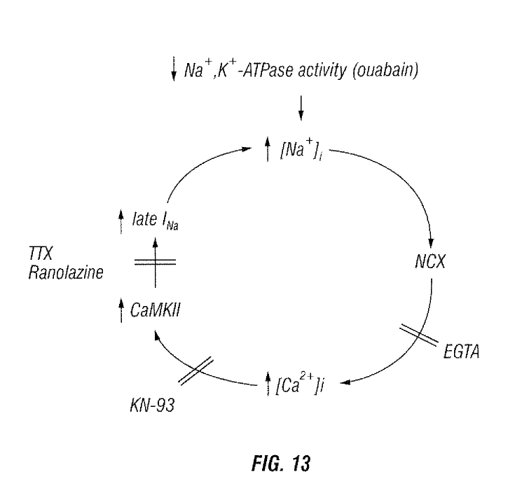

A novel finding in this study is that ouabain increased late I_{Na} in guinea pig isolated ventricular myocytes. Ranolazine, TTX, the CaMKII inhibitor KN-93, and the Ca²⁺-chelator EGTA all reduced late I_{Na} in the presence of ouabain (FIGS. 9, 10). One interpretation of these findings is that the glycoside-induced increase in [Na⁺]ᵢ led to an increase of [Ca²⁺]ᵢ, which led to activation of CaMKII and CaMKII-dependent phosphorylation of the sodium channel, and thereby augmentation of late I_{Na} (FIG. 13). This interpretation is supported by previous results indicating that glycosides increase [Na⁺]ᵢ and [Ca²⁺]ᵢ in the heart, and that Ca²⁺ and CaMKII may directly regulate the function of the cardiac Na⁺ channel to increase late I_{Na}. (Maltsev et al. Am J Physiol Heart Circ Physiol. 2008, 294:H1597-1608, and Biswas et al. Circ Res. 2009; 104:870-878). An increase of late I_{Na} itself leads to Ca²⁺ overload, Maier et al. Cardiovasc Res. 2007; 73:631-640 and Haigney et al. Circulation. 1994; 90:391-399, to close a positive feedback loop between increases of Ca²⁺ and late I_{Na} (FIG. 13). Ranolazine and TTX reduced late I_{Na} and therefore the increase of [Na]ᵢ and the mechanical and electrical dysfunction caused by ouabain. The effect of ouabain on cardiac Na⁺ homeostasis and function therefore has two components: first, the rise of [Na]ᵢ caused by inhibition of Na⁺, K⁺-ATPase and a decreased Na⁺ efflux, and second, the rise of [Na]ᵢ caused by an enhanced late I_{Na} and increased Na⁺ influx. The latter response, which appeared in this study to contribute to cellular Na⁺ and Ca²⁺ overloading and subsequent dysfunction, can be diminished by a late I_{Na} inhibitor, or by inhibition of CaMKII, or by reducing the Ca²⁺ overload (e.g., with EGTA) to prevent the CaMKII-induced enhancement of late I_{Na} (FIG. 13).

In addition to ranolazine and TTX, the putative late I_{Na} inhibitor R56865 is reported to reduce sodium and calcium overload and has beneficial effects (e.g., anti-arrhythmic and reduction of contracture) during exposure of cardiac tissues to cardiac glycosides. (Heers et al. Br J. Pharmacol. 1991; 102:675-678 and Damiano et al. J Cardiovasc Pharinacol. 1991; 18:415-428). Inhibition of NCX in isolated hearts exposed to ouabain has also been shown to reduce Ca²⁺ overload pathology (Imahashi et al. Circ Res. 2005; 97:916-921 and Watarto et al. Br J. Pharmacol. 1999; 127:1846-1850). Taken together, these results indicate that strategies to prevent the pathological increase in late I_{Na} may be cardioprotective.

### Changes in [Na⁺]ᵢ During Sodium Pump Inhibition in the Absence and Presence of Ranotazine and TTX

In many mammals, the [Na⁺]ᵢ in resting heart cells is in the range of 4-8 mmol/L, see, Bers et al. Cardiovasc Res. 2003; 57:897-912. In this study using ²³Na NMR spectroscopy, [Na⁺]ᵢ was found to be 6.9±0.2 mmol/L [Na⁺]ᵢ in guinea pig isolated hearts paced at 5 Hz, consistent with literature reports (Hotta et al. J Cardiovasc Pharmacol. 1998; 31:146-156 and Jelicks et al. Am J. Hypertens. 1995; 8:934-943). Treatment of hearts with 10 μmol/L ranolazine for up to 30 min or with 1 μmol/L TTX for 10 min did not significantly change [Na⁺]ᵢ (FIG. 8B, 8D, 8E). This finding suggests that physiological late I_{Na} is either a small contributor to sodium entry in the beating isolated heart, or that Na⁺ influx due to late I_{Na} does not lead to elevation of [Na⁺]ᵢ because the capacity of the Na⁺, K⁺-ATPase to extrude Na⁺ from the cell exceeds Na⁺ entry, (Akera et al. Life Sci. 1991; 48:97-106) or both. Ranolazine (10 μmol/L) and TTX (1 μmol/L) significantly attenuated the increase of [Na⁺]ᵢ caused by ouabain (FIG. 8D, 8E), suggesting that an enhancement of persistent Na⁺ current (late I_{Na}) by ouabain was a major factor contributing to the increase of [Na⁺]ᵢ. However, ranolazine (10 μmol/L, 30 min; FIG. 8C) did not significantly reduce the level of [Na⁺]ᵢ in hearts previously exposed to ouabain alone for 40 min. This result is not unexpected. Once the cell is “loaded” with Na⁺, Na⁺ extrusion in the presence of ouabain is inhibited and a return of [Na⁺]ᵢ to baseline may be slow, even when Na⁺ entry is reduced. Furthermore, sodium efflux has not been shown to be facilitated by inhibition of late I_{Na} nor by ranolazine, which itself did not alter the activity of the Na⁺, K⁺-ATPase.

### Changes in High-Energy Related Phosphates and Contractility During Sodium Pump Inhibition in the Absence and Presence of I_{NaL} Inhibitors

By inducing increases of [Na⁺]ᵢ and [Ca²⁺]ᵢ, ouabain (0.75 μmol/L) had a positive inotropic effect in the guinea pig isolated heart and papillary muscle preparation. This effect was transient and was followed by mechanical and electrical dysfunction, including a rise of LVEDP, a decrease in LV systolic function, and episodes of cardiac standstill (contracture, inexcitability). Sodium-induced calcium overload is known to lead to a mismatch of energy demand and supply in the heart (Hotta et al. J Cardiovasc Pharmacol. 1998; 31:146-156 and O'Rourke et al. Drug Discov Today Dis Models. 2007; 4:207-217). Energy demand increases due to activation of myosin ATPase, sarcoplasmatic reticulum (SR) Ca²⁺ ATPase, and the sarcolemmal Ca²⁺ ATPase. ATP synthesis may be reduced due to Na⁺ and Ca²⁺ overload (O'Rourke et al. Drug Discov Today Dis Models. 2007; 4:207-217 and Balaban et al. J Mol Cell Cardiol. 2002; 34:1259-1271.37). The mismatch of energy demand and supply results in decreases in [ATP] and [PCr], increases in [ADP], [Pi], and cellular acidosis, and ultimately in decreases in free energy released from ATP hydrolysis, |□G_{˜ATP}| (as absolute value).

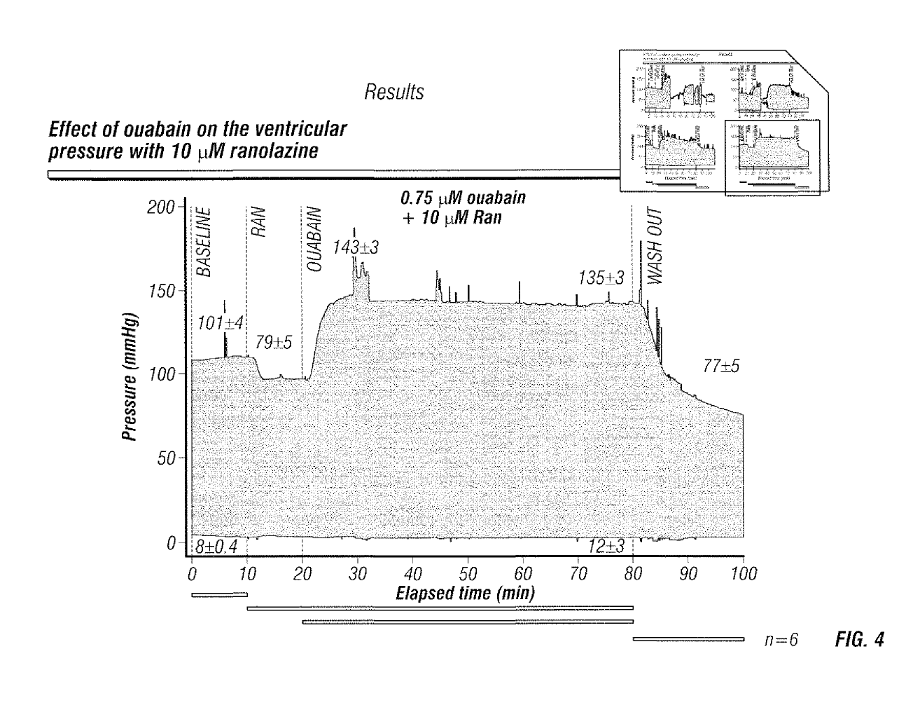

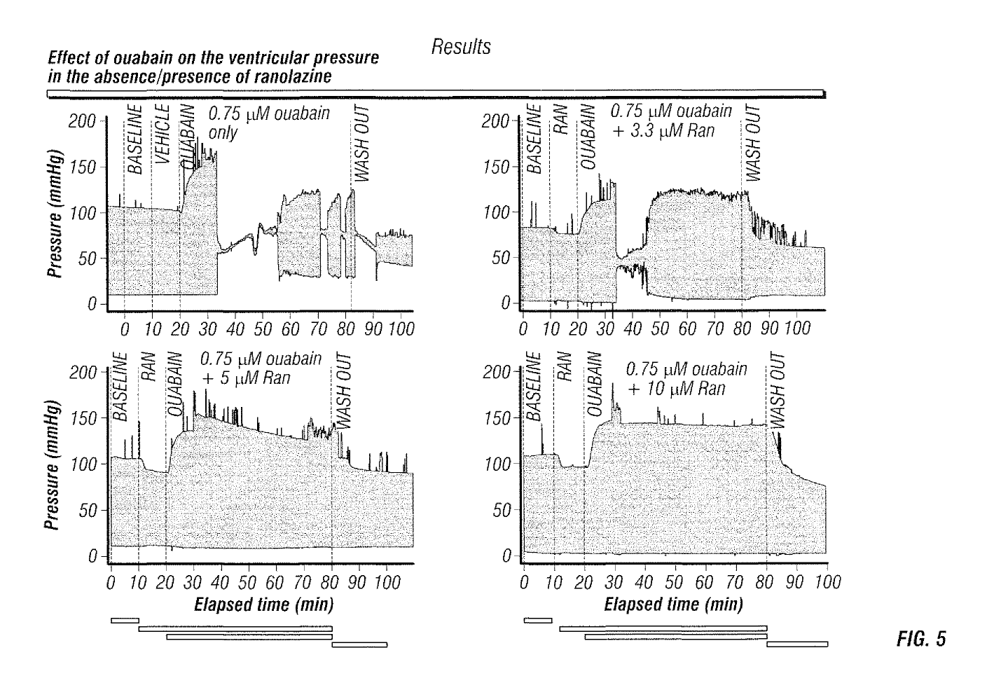

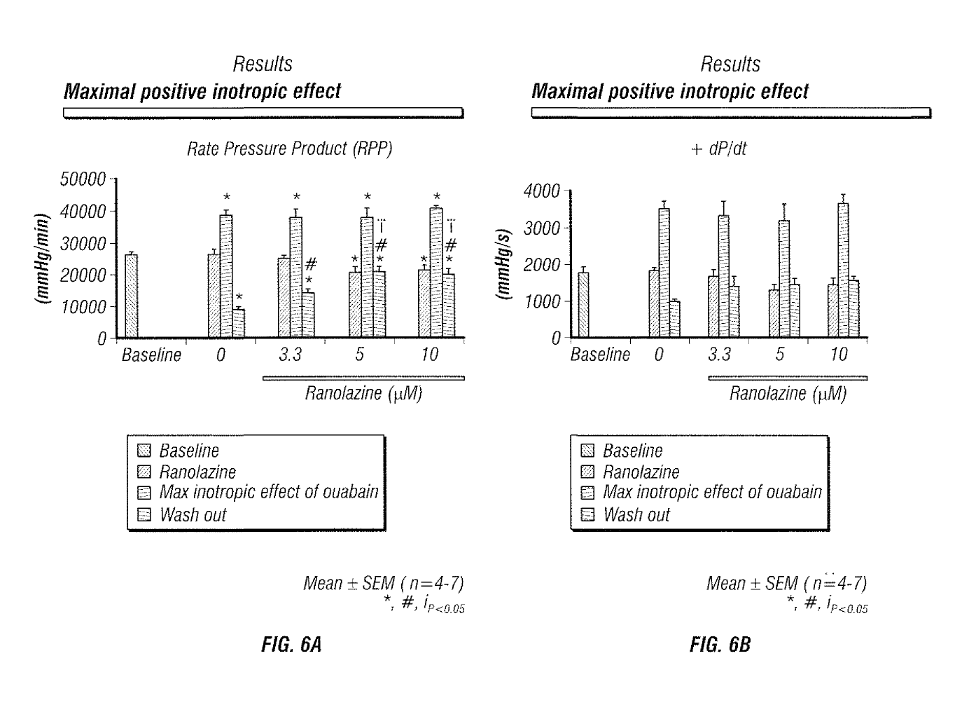

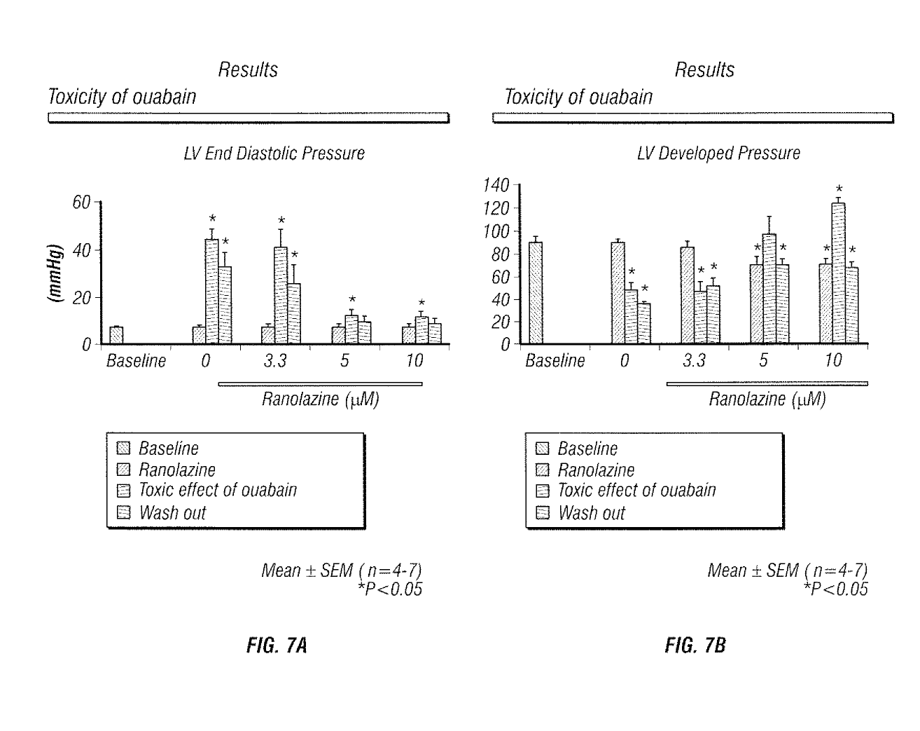

A pronounced loss of over 50% of [ATP] and [PCr], and a decrease of were observed after ouabain treatment in this study. Similar 42 and 66% reductions of [ATP] and [PCr] were reported by Lee et al. (J Pharmacol Exp Ther. 1960; 129:115-122) in a study of cat papillary muscles exposed to 1.37 μmol/L ouabain. Net hydrolysis of ATP and PCr in our study led to a reduction by 7 kJ/mol of the value of |□G_{˜ATP}| to 53-52 kJ/mol (FIG. 11). This value is near the reported value of |□G_{˜ATP}| of 52 kJ/mol needed for operation of the SR calcium pump, Jansen et al. Am J Physiol Heart Circ Physiol. 2003; 285:H2437-2445. A reduction of the value of |□G_{˜ATP}| below this threshold would be predicted to reduce SR calcium uptake and release, and systolic contraction, as was observed in this study (FIG. 5). Ranolazine (10 μmol/L) significantly attenuated the ouabain-induced energy loss and acidosis in the heart (FIG. 4) and, in a concentration-dependent manner, prevented the decrease of LVSP and increase of LVEDP observed during continued exposure of the heart to ouabain (FIG. 12).

We Claim:

1. A method for reducing the toxicity of cardiac glycosides comprising by the co-administration of a therapeutically effective amount of ranolazine to a mammal in need thereof.

2. The method of claim 1, wherein the ranolazine and the cardiac glycoside are administered as separate dosage forms.

3. The method of claim 1, wherein ranolazine and the cardiac glycoside are administered as a single dosage form.

4. The method of claim 1, wherein the cardiac glycoside is selected from the group consisting of digoxin, oubain, digitoxin, and oleandrin.

5. The method of claim 1, wherein the ranolazine and the cardiac glycoside are administered as separate dosage forms.

6. The method of claim 1, wherein ranolazine and the cardiac glycoside are administered as a single dosage form.

7. A method for reducing the undesirable side effects of cardiac glycosides comprising by the co-administration of a therapeutically effective amount of ranolazine to a mammal in need thereof.

8. The method of claim 1, wherein the ranolazine and the cardiac glycoside are administered as separate dosage forms.

9. The method of claim 1, wherein ranolazine and the cardiac glycoside are administered as a single dosage form.

10. The method of claim 1, wherein the cardiac glycoside is selected from the group consisting of digoxin, oubain, digitoxin, and oleandrin.

11. The method of claim 1, wherein the ranolazine and the cardiac glycoside are administered as separate dosage forms.

12. The method of claim 1, wherein ranolazine and the cardiac glycoside are administered as a single dosage form.

13. A pharmaceutical formulation comprising a therapeutically effective amount of ranolazine, a therapeutically effective amount at least one cardiac glycosides, and at least one pharmaceutically acceptable carrier.
